(19) United States
(12) Patent Application Publication
(10) Pub. No.: US 2013/0196935 A1
(43) Pub. Date: Aug. 1, 2013

(54) SYNTHETIC GLYCOAMINE COMPOUNDS

(71) Applicant: Animal Cell Therapies Inc., San Diego, CA (US)

(72) Inventors: Yuefen Zhou, San Diego, CA (US); Kathryn J. Petrucci, La Jolla, CA (US); Xueliang Tao, San Diego, CA (US)

(73) Assignee: ANIMAL CELL THERAPIES INC., San Diego, CA (US)

(21) Appl. No.: 13/751,581

(22) Filed: Jan. 28, 2013

Related U.S. Application Data
(60) Provisional application No. 61/591,603, filed on Jan. 27, 2012.

(51) Int. Cl. C07H 7/04; A61K 45/06; A61K 31/7012

(52) U.S. Cl. CPC C07H 7/04; A61K 31/7012; A61K 45/06
USPC 514/23; 536/18.7

(57) ABSTRACT

This disclosure is directed to synthetic glycoamine compounds and pharmaceutical compositions containing such compounds. The synthetic glycoamine compounds provided here can affect cell adhesion and induce apoptosis, and are useful in treating metastatic diseases and cancer.

### CLAIM OF PRIORITY

This application claims priority under 35 USC §119(e) to U.S. Provisional Application Ser. No. 61/591,603, filed on Jan. 27, 2012, the entire contents of which are hereby incorporated by reference.

### TECHNICAL FIELD

This disclosure is directed to synthetic glycoamine compounds and pharmaceutical compositions containing such compounds. The synthetic glycoamine compounds provided here can affect cell adhesion and induce apoptosis, and are useful in treating metastatic diseases and cancer.

### BACKGROUND

At present, there are limited therapies for cancer patients with advanced metastatic disease. Angiosarcoma (ASA) in humans and hemangiosarcoma (HSA) in dogs are deadly neoplastic diseases characterized by an aggressive growth of malignant cells with endothelial phenotype, widespread metastasis, and poor response to chemotherapy.

Studies in recent years have shown that galectin-3 plays an important role in the biology of ASA and identified Galectin-3 as a potential therapeutic target in tumors arising from malignant endothelial cells. A number of galectin-3 inhibitors have been identified and some of them have been reported to show anti-tumor activity in vivo. However, inhibitors of galectin-3 with improved affinity and pharmacological properties are more desirable, and are in considerable need.

### SUMMARY

This disclosure provides novel synthetic glycoamine compounds and pharmaceutically acceptable salts thereof. These compounds (e.g., a compound of Formula I) are useful in treating metastatic diseases and cancer in a patient in need thereof. For example, a metastatic disease or cancer can be treated in a patient by administering to the patient a therapeutically effective amount of a synthetic glucoamine compound or a pharmaceutically acceptable salt thereof as provided herein.

In a general aspect, provided herein are compounds of Formula I:

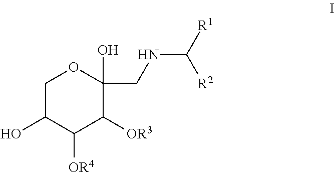

or a pharmaceutically acceptable salt thereof,
wherein:
R¹ is selected from the group consisting of: H, CO₂H, C(O)NH₂, C(O)NHOH, C(O)NHOR⁵, CO₂R⁶, C(O)NHR⁷, C(O)NR⁸R⁹, heterocyclyl, and heteroaryl, wherein R⁵, R⁶, R⁷, R⁸, and R⁹ are independently selected from the group consisting of: C₁-C₆ alkyl, carbocyclyl, heterocyclyl, aryl, and heteroaryl, or R⁸ and R⁹ can combine with the N atom to which they are attached to form a 5 or 6-membered ring, or NHR⁷ is a normatural α-amino acid or a normatural peptide;
R² is selected from the group consisting of: C₁-C₆ alkyl, C₁-C₆ haloalkyl, C₁-C₆ hydroxyalkyl, C₁-C₆ alkoxy, C₂-C₆ alkenyl, C₂-C₆ alkynyl, C₃-C₈ carbocyclyl, heterocyclyl, aryl, and heteroaryl;
   wherein if R¹ is CO₂H, then the —NHCH(R²)CO₂H moiety on the compound of Formula I forms a normatural α-amino acid;
   wherein if R¹ is H, then R² is selected from the group consisting of C₃-C₈ carbocyclyl, benzyl, heterocyclyl, aryl, and heteroaryl;
   wherein the above alkyl, alkenyl, alkynyl, carbocyclyl, heterocyclyl, benzyl, aryl, and heteroaryl moieties are each optionally and independently substituted by 1-3 substituents selected from the group consisting of: amino, cyano, halo, hydroxyl, nitro, C₁-C₆ alkylamine, C₁-C₆ dialkylamine, C₁-C₆ alkyl, C₁-C₆ alkoxy, C₁-C₆ alkenyl, and C₁-C₆ hydroxyalkyl;
   R³ and R⁴ are each independently selected from H and a monosaccharide, provided only one of R³ and R⁴ can be a monosaccharide.

The carbohydrate unit of the Formula I:

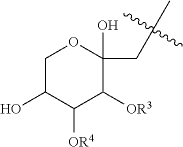

can be a natural or modified sugar. For example, a monosaccharide can be arabinose, xylose, ribose, ribulose, fructose, deoxyfructose, galactose, glucose, mannose, tagatose, rhamnose, or a disaccharide such as lactulose, lactose, maltulose, or maltose. In some embodiments, one or more of the hydroxyl groups on the monosaccharide or disaccharide may be independently protected. For example, a hydroxyl group can be protected with a group such as OAc or another known protecting group.

The Formula I compounds may exist as single stereoisomers (i.e., essentially free of other stereoisomers), racemates, and/or mixtures of enantiomers and/or diastereomers, and tautomers. In some embodiments, the compounds provided herein that are optically active are used in optically pure form.

In some embodiments, R¹ is selected from the group consisting of: H, CO₂H, C(O)NH₂, C(O)NHOH, C(O)NHOR⁵, CO₂R⁶, C(O)NHR⁷, C(O)NR⁸R⁹, heterocyclyl, and heteroaryl; wherein R⁵, R⁶, R⁷, R⁸, and R⁹ are independently selected from the group consisting of C₁-C₆ alkyl, carbocyclyl, heterocyclyl, aryl, and heteroaryl.

In another embodiment, R¹ is C(O)NHR⁷ wherein NHR⁷ is a normatural α-amino acid or a normatural peptide.

In another embodiment, R¹ is selected from the group consisting of: CO₂H, CO₂Me, CO₂Et, C(O)NH₂, C(O)NHOH, C(O)NHMe, and C(O)NH(Me)₂. In some embodiments, R¹ is CO₂H.

In one embodiment, R² is selected from the group consisting of C₁-C₆ alkyl, C₂-C₆ alkenyl, C₂-C₆ alkynyl, C₁-C₆ haloalkyl, C₃-C₈ carbocyclyl, heterocyclyl, aryl, and heteroaryl.

In another embodiment, R² is selected from the group consisting of C₁-C₆ alkyl, C₃-C₈ carbocyclyl, heterocyclyl, aryl and heteroaryl.

In another embodiment, R¹ is H, R² is a C₃-C₈ carbocyclyl, a substituted or unsubstituted benzyl, heterocyclyl, aryl or heteroaryl.

In some embodiments, R¹ is CO₂H, and the —NHCH(R²)CO₂H moiety on the compound of Formula I forms a normatural α-amino acid. For example, R² can be selected from the group consisting of:

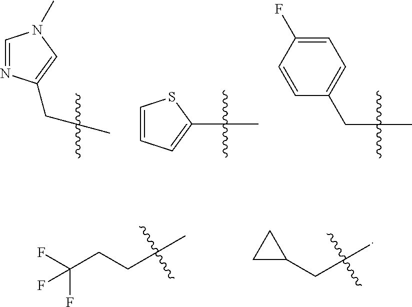

Non-limiting examples of a compound of Formula I include:
3-(3-Methyl-3H-imidazol-4-yl)-2-{[2,3,5-tri hydroxy-4-(3,4,5-trihydroxy-6-hydroxymethyl-tetrahydro-pyran-2-yloxy)-tetrahydro-pyran-2-ylmethyl]-amino}-propionic acid;

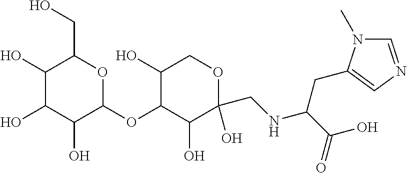 [SMILES: Cn1cncc1CC(NCC1(O)OCC(O)C(OC2OC(CO)C(O)C(O)C2O)C1O)C(=O)O]

Thiophen-2-yl-{[2,3,5-trihydroxy-4-(3,4,5-trihydroxy-6-hydroxymethyl-tetrahydro-pyran-2-yloxy)-tetrahydro-pyran-2-ylmethyl]-amino}-acetic acid;

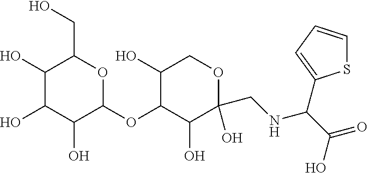 [SMILES: O=C(O)C(NCC1(O)OCC(O)C(OC2OC(CO)C(O)C(O)C2O)C1O)c1cccs1]

3-(4-Fluoro-phenyl)-2-{[2,3,5-trihydroxy-4-(3,4,5-trihydroxy-6-hydroxymethyl-tetrahydro-pyran-2-yloxy)-tetrahydro-pyran-2-ylmethyl]-amino}-propionic acid;

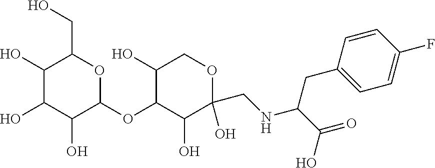 [SMILES: O=C(O)C(Cc1ccc(F)cc1)NCC1(O)OCC(O)C(OC2OC(CO)C(O)C(O)C2O)C1O]

5,5,5-Trifluoro-2-{[2,3,5-trihydroxy-4-(3,4,5-trihydroxy-6-hydroxymethyl-tetrahydro-pyran-2-yloxy)-tetrahydro-pyran-2-ylmethyl]-amino}-pentanoic acid;

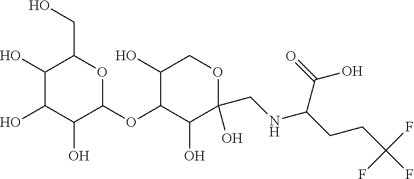 [SMILES: O=C(O)C(CCC(F)(F)F)NCC1(O)OCC(O)C(OC2OC(CO)C(O)C(O)C2O)C1O]

3-Cyclopropyl-2-{[2,3,5-tri hydroxy-4-(3,4,5-trihydroxy-6-hydroxymethyl-tetrahydro-pyran-2-yloxy)-tetrahydro-pyran-2-ylmethyl]-amino}-propionic acid;

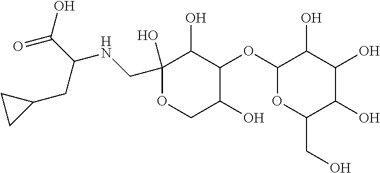 [SMILES: O=C(O)C(CC1CC1)NCC1(O)OCC(O)C(OC2OC(CO)C(O)C(O)C2O)C1O]

3-Cyclopropyl-2-{[2,3,5-tri hydroxy-4-(3,4,5-trihydroxy-6-hydroxymethyl-tetrahydro-pyran-2-yloxy)-tetrahydro-pyran-2-ylmethyl]-amino}-propionic acid methyl ester;

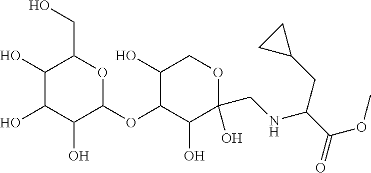 [SMILES: COC(=O)C(CC1CC1)NCC1(O)OCC(O)C(OC2OC(CO)C(O)C(O)C2O)C1O]

3-Cyclopropyl-2-{[2,3,5-tri hydroxy-4-(3,4,5-trihydroxy-6-hydroxymethyl-tetrahydro-pyran-2-yloxy)-tetrahydro-pyran-2-ylmethyl]-amino}-propionamide;

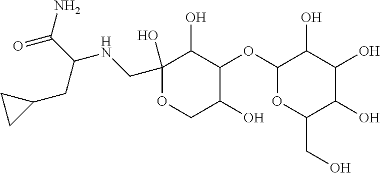 [SMILES: NC(=O)C(CC1CC1)NCC1(O)OCC(O)C(OC2OC(CO)C(O)C(O)C2O)C1O]

(4-Fluoro-phenyl)-{[2,3,5-trihydroxy-4-(3,4,5-trihydroxy-6-hydroxymethyl-tetrahydro-pyran-2-yloxy)-tetrahydro-pyran-2-ylmethyl]-amino}-methane;

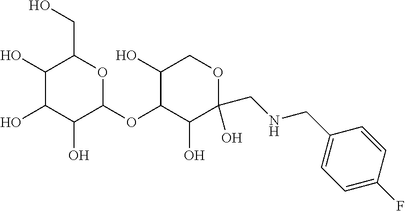 [SMILES: OCC1OC(OC2C(O)COC(O)(CNCc3ccc(F)cc3)C2O)C(O)C(O)C1O]

Cyclopropyl-{[2,3,5-tri hydroxy-4-(3,4,5-trihydroxy-6-hydroxymethyl-tetrahydro-pyran-2-yloxy)-tetrahydro-pyran-2-ylmethyl]-amino}-ethane

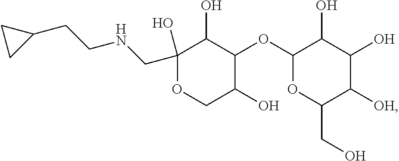 [SMILES: OCC1OC(OC2C(O)COC(O)(CNCCC3CC3)C2O)C(O)C(O)C1O]

or a pharmaceutically acceptable salt thereof.

Further provided herein are pharmaceutically acceptable salts of a compound of Formula I and pharmaceutical compositions comprising the same. A method of making a compound of Formula I is also provided.

Also provided herein is a method for treating metastatic diseases and cancer in a patient in need thereof. In some embodiments, a method comprises administering to the patient a therapeutically effective amount of a Formula I compound or a pharmaceutically acceptable salt thereof. In one embodiment, a method for treating metastatic diseases and cancer in a patient in need thereof is provided, comprising administering to the patient a therapeutically effective amount of a Formula I compound or a pharmaceutically acceptable salt thereof that is an inhibitor of galectin-3.

In another embodiment, a method for treating metastatic diseases and cancer in a patient in need thereof is provided, comprising administering to the patient a therapeutically effective amount of a pharmaceutical composition comprising a compound of Formula I or a pharmaceutically acceptable salt thereof and a pharmaceutically acceptable excipient, carrier, or vehicle.

Further provided herein is a method for treating metastatic diseases and cancer in a patient in need thereof, comprising administering to the patient a therapeutically effective amount of a compound of Formula I and an additional therapeutic agent, for example, an anti-cancer agent.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows the SVR cell colony stained with hematoxylin (100× magnification).

FIG. 2 is a line graph comparing the effects of ACT-1 and ACT-2 on the clonogenic survival of SVR cells.

FIG. 3 is a line graph comparing the effects of ACT-1 and ACT-2 on the clonogenic survival of SVR cells.

FIG. 4 shows the results of the TUNEL assay on SVR cells treated with ACT-1 and ACT-2.

FIG. 5 is a line drawing illustrating the cytotoxic effect of ACT-1 and ACT-2 on BAEC cells.

FIG. 6 is a line drawing illustrating the cytotoxic effect of ACT-1 and ACT-2 on SVR cells.

### DETAILED DESCRIPTION OF THE INVENTION

Where the following terms are used in this specification, they are used as defined below:

The terms “comprising,” “having” and “including” are used herein in their open, non-limiting sense.

The term “alkyl”, as used herein, unless otherwise indicated, includes saturated monovalent hydrocarbon radicals having straight or branched, or a combination of the foregoing moieties.

The term “alkenyl”, as used herein, unless otherwise indicated, includes alkyl moieties having at least one carbon-carbon double bond wherein alkyl is as defined above and including E and Z isomers of said alkenyl moiety.

The term “alkynyl”, as used herein, unless otherwise indicated, includes alkyl moieties having at least one carbon-carbon triple bond wherein alkyl is as defined above.

The term “alkoxy”, as used herein, unless otherwise indicated, includes O-alkyl groups wherein alkyl is as defined above.

The term “Me” means methyl, “Et” means ethyl, and “Ac” means acetyl.

The term “carbocyclyl”, as used herein, unless otherwise indicated refers to a non-aromatic, saturated or partially saturated, monocyclic or fused, spiro or unfused bicyclic or tricyclic hydrocarbon ring referred to herein as containing a total of from 3 to 10 carbon atoms (e.g., 5-8 ring carbon atoms). Exemplary carbocyclyls include monocyclic rings having from 3-7, e.g., 3-6, carbon atoms, such as cyclopropyl, cyclobutyl, cyclopentyl, cyclohexyl, cycloheptyl and the like.

The term “aryl”, as used herein, unless otherwise indicated, includes an organic radical derived from an aromatic hydrocarbon by removal of one hydrogen, such as phenyl or naphthyl.

The term “heterocyclyl”, as used herein, unless otherwise indicated, includes a stable, mono- or multi-cyclic non-aromatic heterocyclic ring system which consists of carbon atoms and at least one heteroatom selected from the group consisting of N, O, and S, wherein the nitrogen and sulfur heteroatoms may be optionally oxidized, and the nitrogen atom may be optionally quaternized. For example, the ring can have 1, 2, 3 or 4 N, or 1, 2 or 3 O or S atoms. The heterocyclic system may be attached, unless otherwise stated, at any heteroatom or carbon atom which affords a stable structure. Examples of non-aromatic heterocycles include monocyclic groups such as: aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide. Examples of polycyclic heterocycles include: indolinyl, quinolyl, tetrahydroquinolyl, isoquinolyl, particularly 1- and 5-isoquinolyl, 1,2,3,4-tetrahydroisoquinolyl, cinnolinyl, quinoxalinyl, particularly 2- and 5-quinoxalinyl, quinazolinyl, phthalazinyl, 1,5-naphthyridinyl, 1,8-naphthyridinyl, 1,4-benzodioxanyl, dihydrocoumarin, 2,3-dihydrobenzofuryl, 1,2-benzisoxazolyl, benzothienyl, particularly 3-, 4-, 5-, 6-, and 7-benzothienyl, benzoxazolyl, benzthiazolyl, particularly 2-benzothiazolyl and 5-benzothiazolyl, purinyl, benzimidazolyl, particularly 2-benzimidazolyl, benztriazolyl, thioxanthinyl, carbazolyl, carbolinyl, acridinyl, pyrrolizidinyl, and quinolizidinyl.

The term “heteroaryl” as used herein, unless otherwise indicated, refers to a heterocycle having aromatic character. A polycyclic heteroaryl may include one or more rings which are partially saturated. Examples include tetrahydroquinoline and 2,3-dihydrobenzofuryl. Examples of heteroaryl groups include: pyridyl, pyrazinyl, pyrimidinyl, particularly 2- and 4-pyrimidinyl, pyridazinyl, thienyl, furyl, pyrrolyl, particularly 2-pyrrolyl, imidazolyl, thiazolyl, oxazolyl, pyrazolyl, particularly 3- and 5-pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Unless defined otherwise, “alkyl,” “alkylene,” “alkenyl,” “alkynyl,” “aryl,” “carbocyclyl,” and “heterocyclyl” are each optionally and independently substituted by 1-3 substituents selected from alkanoyl, alkylamine, amino, aryl, carbocyclyl, heterocyclyl, C₁-C₆ alkyl, C₁-C₆ haloalkyl, C₁-C₆ hydroxyalkyl, C₁-C₆ alkoxy, C₁-C₆ alkylamine, C₁-C₆ dialkylamine, C₂-C₆ alkenyl, or C₂-C₆ alkynyl, wherein each of which may be interrupted by one or more hetero atoms; carboxyl, cyano, halo, hydroxy, nitro, —C(O)OH, —C(O)₂—(C₁-C₆ alkyl), —C(O)₂—(C₃-C₈ carbocyclyl), —C(O)₂-(aryl), —C(O)₂-(heterocyclyl), —C(O)₂—(C₁-C₆ alkylene)aryl, —C(O)₂—(C₁-C₆ alkylene)heterocyclyl, —C(O)₂—(C₁-C₆ alkylene)carbocyclyl, —C(O)(C₁-C₆ alkylene), —C(O)(C₃-C₈ carbocyclyl), —C(O)(aryl), —C(O)(heterocyclyl), —C(O)(C₁-C₆ alkylene)aryl, —C(O)(C₁-C₆ alkylene)heterocyclyl, and —C(O)(C₁-C₆ alkylene)carbocyclyl.

The term “peptide” means a short polymer of no more than 10 amino acid monomers linked by peptide bonds. Such a polymer may contain natural or normatural amino acid monomers. In some embodiments, the peptide contains at least one normatural amino acid monomers. In some embodiments, the peptide contains all normatural amino acid monomers.

The term “patient” means an animal (e.g., cow, horse, sheep, pig, chicken, turkey, quail, cat, dog, mouse, rat, rabbit, guinea pig, etc.) or a mammal (e.g., a human), including chimeric and transgenic animals and mammals. In some embodiments, in the treatment of cancer, the term “patient” refers to an animal or a human. In a specific embodiment the patient has metastatic cancer.

The term a “therapeutically effective amount” refers to an amount of a compound provided herein sufficient to provide a benefit in the treatment of cancer metastasis, to delay or minimize symptoms associated with metastatic cancer, or to ameliorate a disease or infection or cause thereof. In particular, a therapeutically effective amount means an amount sufficient to provide a therapeutic benefit in vivo. Used in connection with an amount of a compound provided herein, the term can encompass a non-toxic amount that improves overall therapy, reduces or symptoms of a disease, or enhances the therapeutic efficacy of or synergies with another therapeutic agent.

The term “in combination” refers to the use of more than one therapeutic agents simultaneously or sequentially and in a manner that their respective effects are additive or synergistic.

The term “treating” refers to causing a therapeutically beneficial effect, such as ameliorating existing symptoms, ameliorating the underlying metabolic causes of symptoms, postponing or preventing the further development of a disorder and/or reducing the severity of symptoms that will or are expected to develop.

The terms “α” and “β” indicate the specific stereochemical configuration of a substituent at an asymmetric carbon atom in a chemical structure as drawn.

The term “normatural amino acids” refers to the amino acids that are not naturally-occurring amino acids. They are not any of the twenty known natural amino acids including histidine, arginine, lysine, isoleucine, phenylalanine, leucine, tryptophan, alanine, methionine, proline, cysteine, asparagines, valine, glycine, serine, glutamine, tyrosine, aspartic acid, glutamic acid and threonine.

A compound provided herein may exhibit the phenomenon of tautomerism. While Formula I does not expressly depict all possible tautomeric forms, it is to be understood that Formula I is intended to represent any tautomeric form of the depicted compound and is not to be limited merely to a specific compound form depicted by the formula drawings.

Some of the compounds provided herein may exist as single stereoisomers (i.e., essentially free of other stereoisomers), racemates, and/or mixtures of enantiomers and/or diastereomers. All such single stereoisomers, racemates and mixtures thereof are intended to be within the scope of the present disclosure. In some embodiments, a compound provided herein that is optically active is used in its optically pure form.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. In some embodiments, a compound provided herein is used in a form that is at least 90% optically pure, that is, a form that contains at least 90% of a single isomer (80% enantiomeric excess (“e.e.”) or diastereomeric excess (“d.e.”)). For example, at least 95% (90% e.e. or d.e.), at least 97.5% (95% e.e. or d.e.), or at least 99% (98% e.e. or d.e.).

“A pharmaceutically acceptable salt” is intended to mean a salt that retains the biological effectiveness of the free acids and bases of the specified compound and that is not biologically or otherwise undesirable. A compound provided herein may possess a sufficiently acidic, a sufficiently basic, or both functional groups, and accordingly react with any of a number of inorganic or organic bases, and inorganic and organic acids, to form a pharmaceutically acceptable salt.

If a compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an α-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If a compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal or polymorphic forms, all of which are intended to be within the scope of the present invention and specified formulas.

A pharmaceutical composition comprising a compound of Formula I may be adapted for oral, intravenous, intramuscular, topical, intraperitoneal, nasal, buccal, sublingual, or subcutaneous administration, or for administration via respiratory tract in the form of, for example, an aerosol or an air-suspended fine powder.

The dosage of a compound of Formula I may vary depending on the route of administration, individual body weight and age, as well as the condition of the disease.

A pharmaceutical composition provided herein may optionally comprise two or more compounds of the Formula I without an additional therapeutic agent.

In some embodiments, a method provided herein includes the administration of an additional therapeutic agent (i.e., a therapeutic agent other than a compound provided herein). For example, the compounds of the invention can be used in combination with at least one other therapeutic agent. Therapeutic agents include, but are not limited to antibiotics, antiemetic agents, antidepressants, and antifungal agents, anti-inflammatory agents, antiviral agents, and anticancer agents. Examples of anticancer agents include: doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, homoharringtonin, hydroxyurea, idarubicin, ifosfamide, irinotecan, lomustine, melphalan, mercaptopurine, methotrexate, mitomycin, mitoxantron, mitozantrone, oxaliplatin, paclitaxel, procarbazine, rituxan, Schisandrin B, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof. In some embodiments, the additional therapeutic agent is an anti-cancer agent, for example, paclitaxel.

A compound provided herein in combination with another therapeutic agent can act additively or synergistically. In one embodiment, a composition comprising a compound provided herein is administered concurrently with the administration of another therapeutic agent, which can be part of the same composition or in a different composition from that comprising a compound provided herein. In another embodiment, a compound provided herein is administered prior to or subsequent to administration of another therapeutic agent.

### Preparation of Compounds

In the synthetic schemes described below, unless otherwise indicated, all temperatures are set forth in degrees Celsius and all parts and percentages are by weight.

Reagents purchased from commercial suppliers are used without further purification unless otherwise indicated. All solvents purchased from commercial suppliers are used as received.

The reactions set forth below are done or can be done generally under a positive pressure of argon or nitrogen at an ambient temperature (unless otherwise stated) in anhydrous solvents, and the reaction flasks are fitted with rubber septa for the introduction of substrates and reagents via syringe. Glassware is oven dried and/or heat dried.

The reactions are assayed by TLC and/or analyzed by LC-MS and terminated as judged by the consumption of starting material. Analytical thin layer chromatography (TLC) is performed on glass-plates precoated with silica gel 60 F₂₅₄ 0.25 mm plates, and visualized with UV light (254 nm) and/or iodine on silica gel and/or heating with TLC stains such as ethanolic phosphomolybdic acid, ninhydrin solution, potassium permanganate solution or ceric sulfate solution. Preparative thin layer chromatography (prep TLC) is performed on glass-plates precoated with silica gel 60 F₂₅₄ 0.5 mm plates and visualized with UV light (254 nm).

Work-ups are typically done by doubling the reaction volume with the reaction solvent or extraction solvent and then washing with the indicated aqueous solutions using 25% by volume of the extraction volume unless otherwise indicated. Product solutions are dried over anhydrous Na₂SO₄ and/or MgSO₄ prior to filtration and evaporation of the solvents under reduced pressure on a rotary evaporator and noted as solvents removed in vacuo. Column chromatography is completed under positive pressure using silica gel 230-400 mesh or 50-200 mesh neutral alumina, or on silica gel columns. Hydrogenolysis is done at the pressure indicated in the examples or at ambient pressure.

¹H-NMR spectra and ¹³C-NMR are recorded on a Varian Mercury-VX400 instrument operating at 400 MHz. NMR spectra are obtained as CDCl₃ solutions (reported in ppm), using chloroform as the reference standard (7.27 ppm for the proton and 77.00 ppm for carbon), CD₃OD (3.4 and 4.8 ppm for the protons and 49.3 ppm for carbon), DMSO-d₆ (2.49 ppm for proton), or internally tetramethylsilane (0.00 ppm) when appropriate. Other NMR solvents are used as needed. When peak multiplicities are reported, the following abbreviations are used: s (singlet), d (doublet), t (triplet), q (quartet), m (multiplet), br (broadened), bs (broad singlet), dd (doublet of doublets), dt (doublet of triplets). Coupling constants, when given, are reported in Hertz (Hz).

LC-MS (Mass spectra) are run using (+)- or (−)-ES or APCI (+ or −) method. Melting points (mp) are determined on an open capillary apparatus, and are uncorrected.

The described synthetic pathways and experimental procedures utilize many common chemical abbreviations, 2,2-DMP (2,2-dimethoxypropane), Ac (acetyl), ACN (acetonitrile), Bn (benzyl), BOC (tert-butoxycarbonyl), Bz (benzoyl), DBU (1,8-diazabicyclo[5,4,0]undec-7-ene, DCC(N,N′-dicyclohexylcarbodiimide), DCE (1,2-dichloroethane), DCM (dichloromethane), DEAD (diethylazodicarboxylate), DIEA (diisopropylethylamine), DMA (N,N-dimethylacetamide), DMAP (4-(N,N-dimethylamino)pyridine), DMF (N,N-dimethylformamide), DMSO (dimethyl sulfoxide), EDC (1-(3-dimethylaminopropyl)-3-ethylcarbodiimide hydrochloride), Et (ethyl), EtOAc (ethyl acetate), EtOH (ethanol), HATU (O-(7-azabenzotriazol-1-yl)-1,1,3,3-tetramethyluronium hexafluorophosphate), HBTU (O-benzotriazol-1-yl-N,N,N′,N′-tetramethyluronium hexafluorophosphate), HF (hydrogen fluoride), HOBT (1-hydroxybenzotriazole hydrate), HPLC (high pressure liquid chromatography), IPA (isopropyl alcohol), KO^{t}Bu (potassium tert-butoxide), LDA (lithium diisopropylamine), MCPBA (3-chloroperbenzoic acid), Me (methyl), MeCN (acetonitrile), MeOH (methanol), NaH (sodium hydride), NaOAc (sodium acetate), NaOEt (sodium ethoxide), Phe (phenylalanine), PPTS (pyridinium p-toluenesulfonate), PS (polymer supported), Py (pyridine), pyBOP (benzotriazol-1-yloxy)tripyrrolidinophosphonium hexafluorophosphate), TEA (triethylamine), TFA (trifluoroacetic acid), TFAA (trifluoroacetic anhydride), THF (tetrahydrofuran), TLC (thin layer chromatography), Tol (toluoyl), Val (valine), H⁺ (any acid) and the like.

Scheme 1 provides a general method that can be used to prepare compounds of Formula I.

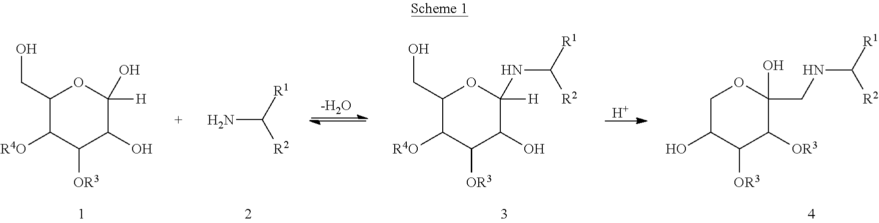

In a general method, a sugar 1 (e.g., a monosaccharide or a disaccharide) can be treated with an amino compound 2 in a solvent or co-solvents such as methanol and glycerol under heating to give a Schiff base 3, which undergoes rapid rearrangement under acidic condition and heating to a glycoamine 4.

### Example 1

### Scheme 2 Describes the Synthesis of Compound 4a

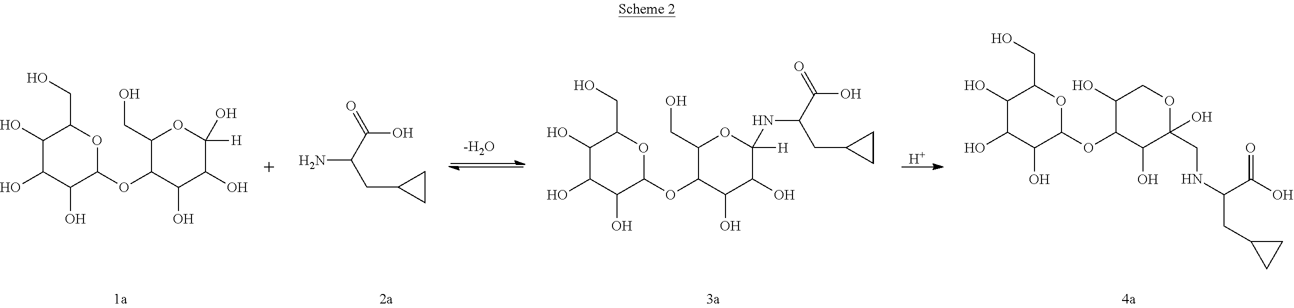

In Scheme 2, a lactose (1a) can be reacted with a normatural amino acid of 2-amino-3-cyclopropyl-propionic acid (2a) in MeOH and glycerol under heating condition, e.g., reflux condition, to form a Schiff base 3a, which undergoes rapid rearrangement under acidic condition such as acetic acid and heating, e.g., reflux condition, to the desired product of 3-cyclopropyl-2-{[2,3,5-trihydroxy-4-(3,4,5-trihydroxy-6-hydroxymethyl-tetrahydro-pyran-2-yloxy)-tetrahydro-pyran-2-ylmethyl]-amino}-propionic acid (4a). The compound 4a thus prepared can be purified on a column of an ion-exchange resin such as Dowex (H⁺ form) and IRN-77 (hydrogen form) for biological and pharmacological evaluation. Compounds 4a are also known as Amadori compounds.

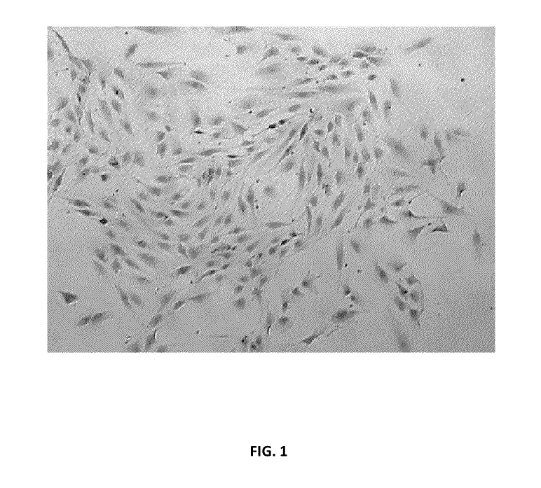

By using D-lactose, and racemic 2-amino-3-cyclopropyl-propionic acid, or its D- or L-form of 2a, with the method described in Scheme 2, 4a with various configurations such as 4a-1-4-a-6 may be obtained as shown in FIG. 1. They may be isolated by chiral separation of the racemic 4a.

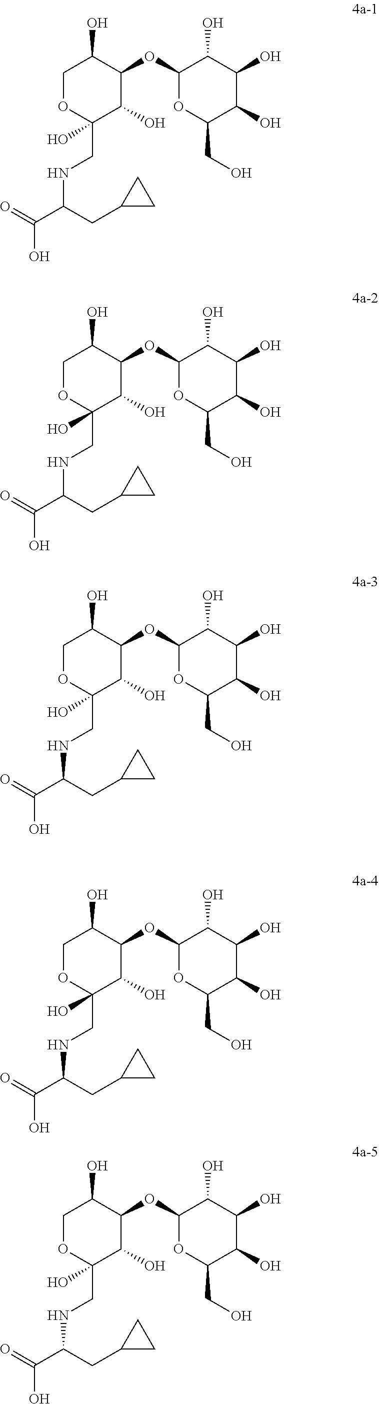 [SMILES: O=C(O)C(CC1CC1)NC[C@@]1(O)OC[C@@H](O)[C@@H](O[C@@H]2O[C@H](CO)[C@H](O)[C@H](O)[C@H]2O)[C@@H]1O ; O=C(O)C(CC1CC1)NC[C@]1(O)OC[C@@H](O)[C@@H](O[C@@H]2O[C@H](CO)[C@H](O)[C@H](O)[C@H]2O)[C@@H]1O ; O=C(O)[C@H](CC1CC1)NC[C@@]1(O)OC[C@@H](O)[C@@H](O[C@@H]2O[C@H](CO)[C@H](O)[C@H](O)[C@H]2O)[C@@H]1O ; O=C(O)[C@H](CC1CC1)NC[C@]1(O)OC[C@@H](O)[C@@H](O[C@@H]2O[C@H](CO)[C@H](O)[C@H](O)[C@H]2O)[C@@H]1O ; O=C(O)[C@@H](CC1CC1)NC[C@@]1(O)OC[C@@H](O)[C@@H](O[C@@H]2O[C@H](CO)[C@H](O)[C@H](O)[C@H]2O)[C@@H]1O]

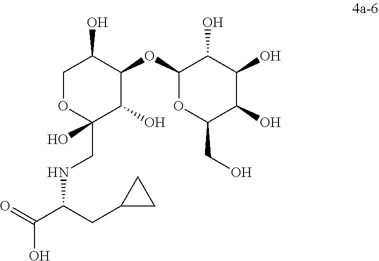 [SMILES: O=C(O)[C@@H](CC1CC1)NC[C@]1(O)OC[C@@H](O)[C@@H](O[C@@H]2O[C@H](CO)[C@H](O)[C@H](O)[C@H]2O)[C@@H]1O]

The Amadori compounds 4 may be exist in their tautomeric forms in aqueous solutions as illustrated below where R═CHR¹R².

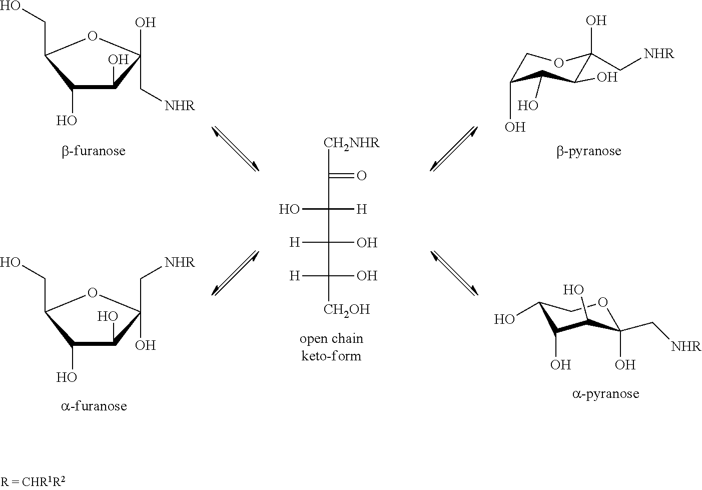

### Biological Testing

The ability of a compound of Formula I to inhibit galectin-3, affect cell adhesion, induce apoptosis, and treat metastatic diseases and cancer can be demonstrated, for example, in the following assays.

### Galectin-3 Inhibition Assay

A compound of Formula I can be evaluated for its efficiency in inhibiting galectin-3 in a known fluorescence polarization-based assay (Sörme, P. et al. Meth. Enzymol. 2003, 362, 504-512). Briefly, to galectin-3 and a suitable fluorescent probe (0.1 μM) in a multiwall plate, the test compound at various concentrations is added, the plate is incubated under slow rotary shaking in the dark for 5 minutes, and fluorescence polarization measured at room temperature. Control wells containing only fluorescent probe are included.

Apoptosis Induction Experiments and Determination of IC-Min and IC-50 of Modified Lactosyl-Leucine (LL) and Modified LL with Doxyrubricin

Apoptosis studies can be performed at various concentrations of modified LL (e.g., a compound of Formula (I)) to determine the IC-min and IC-50 of modified LL using the TdT-mediated deoxyuridine triphosphate nick end labeling (TUNEL) method. Tumor cells, grown until 50% to 60% confluent, can be harvested using a nonenzymatic cell dissociation reagent and pipetted to produce a single-cell suspension. Cells can be plated at low density (200 cells/well) in quadruplicate using four-well chamber slides without the Gal-3 inhibitor tested (control), with the Gal-3 inhibitor tested, with the Gal-3 inhibitor and doxyrubricin, and finally with doxyrubricin alone. After 24 hours, the cells can be fixed in 2% formaldehyde in PBS. TUNEL assays can then be performed using the in situ Cell Death Detection kit POD (Roche Diagnostics, Indianapolis, Ind.) according to the manufacturer's protocol, and apoptotic and nonapoptotic cells will be scored.

Several studies of models of human cancer in mice indicate that enhanced expression of galectin-3 results in faster tumor growth and more metastasis (Bresalier, R. S. et al., Gastroenterology, 1998, 115, 287-296; Leffler, H., Glycoconj. J., 2004, 19, 433-638). Injection of saccharide with inhibitory potency to galectin-3 was reported to diminish prostate cancer in rat (Pienta, K. J., J. Natl. Cancer Inst., 1995, 87, 348-353). It has been reported that some galectin-3 inhibitor increases metastatic cancer cell sensitivity to taxol-induced apoptosis both in vitro and in vivo (Neoplasia, 2009, 11(9), 901-909). Hence, potent small molecule inhibitors of galectin-3 are expected to have similar anticancer effects.

### EXAMPLES

### Example 1

### Preparation of ACT-1 and ACT-2

### General Synthetic Method:

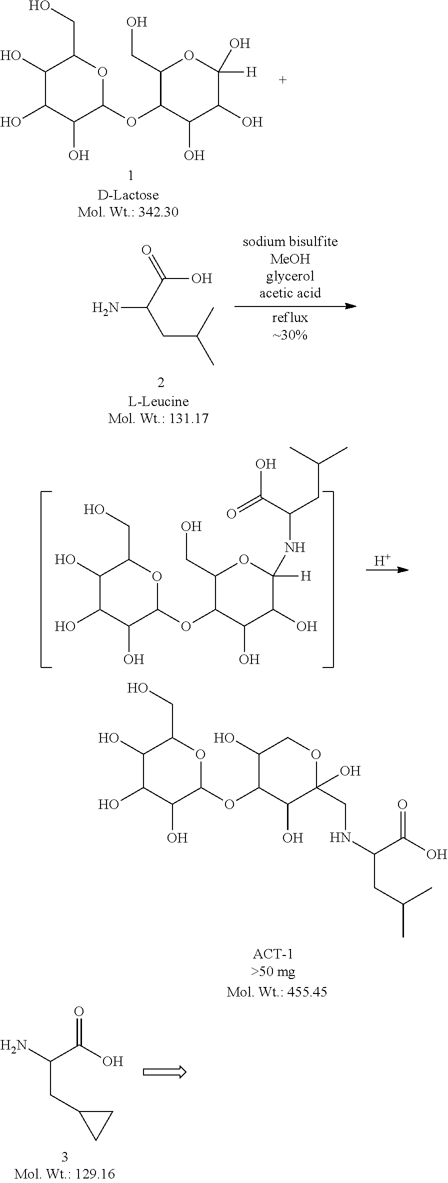

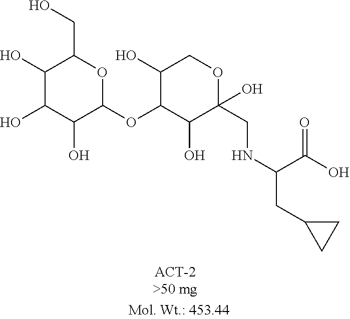 [SMILES: O=C(O)C(CC1CC1)NCC1(O)OCC(O)C(OC2OC(CO)C(O)C(O)C2O)C1O]

### Preparation of Compound 3

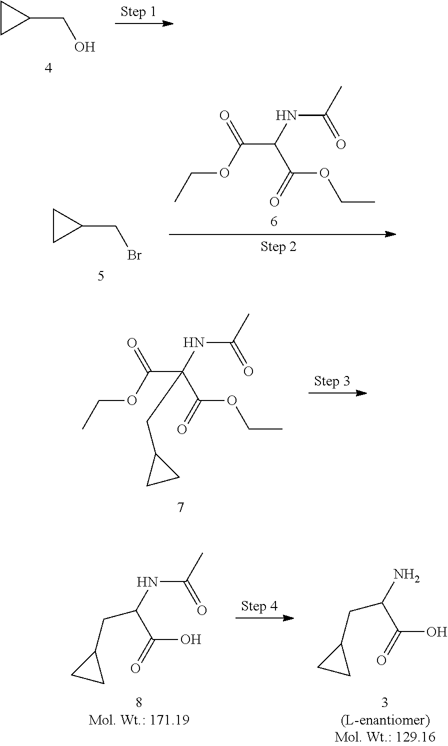 [SMILES: OCC1CC1 ; CCOC(=O)C(NC(C)=O)C(=O)OCC ; BrCC1CC1 ; CCOC(=O)C(CC1CC1)(NC(C)=O)C(=O)OCC ; CC(=O)NC(CC1CC1)C(=O)O ; NC(CC1CC1)C(=O)O]

Analytical data for the prepared compounds:

### ACT-1:

A white solid; LC-MS (ES⁺, m/z): 456.3 [M+1]⁺ (100%); ¹³C-NMR (100 MHz, D₂O), [major peaks, ppm] 176.74, 103.72, 97.94, 79.88, 78.15, 75.32, 73.49, 71.40, 71.34, 69.32, 66.21, 64.94, 63.93, 55.22, 41.66, 27.29, 24.79, 24.02.

### ACT-2:

A white solid; LC-MS (ES⁺, m/z): 454.2 [M+1]⁺ (100%); ¹³C-NMR (100 MHz, D₂O), [major peaks, ppm] 176.05, 103.7, 97.8, 79.92, 78.09, 75.33, 73.47, 71.67, 71.38, 69.19, 66.30, 66.21, 63.84, 55.74, 36.87, 8.61, 6.40, 6.22.

The compounds prepared above had the following stero-configuration:

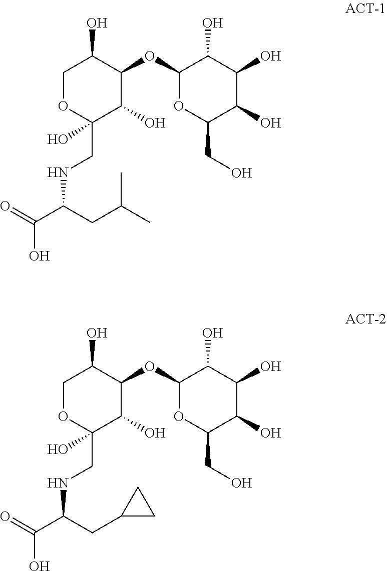 [SMILES: CC(C)C[C@@H](NC[C@@]1(O)OC[C@@H](O)[C@@H](O[C@@H]2O[C@H](CO)[C@H](O)[C@H](O)[C@H]2O)[C@@H]1O)C(=O)O ; O=C(O)[C@H](CC1CC1)NC[C@@]1(O)OC[C@@H](O)[C@@H](O[C@@H]2O[C@H](CO)[C@H](O)[C@H](O)[C@H]2O)[C@@H]1O]

### Example 2

### In Vitro Validation of Efficacy of ACT-2 on SVR Cells with TUNEL and Clonogenic Survival Assays

A. The Effect of ACT-1 and ACT-2 Compounds on the Clonogenic Survival of SVR Cells

### Experimental Procedures:

1. Testing concentrations of ACT-1 and ACT-2: 1 mM, 500 μM, 250 μM, 125 μM, and 62.5 μM.
2. Prepared 2 mM ACT-1=0.91 mg/mL and 2 mM ACT-2=0.91 mg/mL in SVR complete culture medium. Sterilized the test article solutions by filtering through 0.2 μm syringe filters (Whatman Puradisc 25 mm, Cat #6780-2502).
3. Prepared solutions in 2× testing concentrations (2 mM, 1 mM, 500 μM, 250 μM, 125 μM and 62.5 μM) by preparing 2× dilution from 2 mM stock solution (mixing 2.4 mL culture medium with 2.4 mL of 2 mM solution, and make serial 2× dilutions the same way).
4. Pipetted 0.5 mL of test articles to 24-well plates according to the layout below (ACT-1 in one plate and ACT-2 in another plate). Each concentration will be tested in quadruplicate.

TABLE 1
Experimental layout (200 SVR cells per well):

| | 1 | 2 | 3 | 4 | 5 | 6 |
|---|---|---|---|---|---|---|
| A | ACT-1 or ACT-2 | ACT-1 or ACT-2 | ACT-1 or ACT-2 | ACT-1 or ACT-2 | ACT-1 or ACT-2 | ACT-1 or ACT-2 |
| | Medium | 62.5 μM | 125 μM | 250 μM | 500 μM | 1 mM |
| | control | | | | | |
| B | ACT-1 or ACT-2 | ACT-1 or ACT-2 | ACT-1 or ACT-2 | ACT-1 or ACT-2 | ACT-1 or ACT-2 | ACT-1 or ACT-2 |
| | Medium | 62.5 μM | 125 μM | 250 μM | 500 μM | 1 mM |
| | Control | | | | | |
| C | ACT-1 or ACT-2 | ACT-1 or ACT-2 | ACT-1 or ACT-2 | ACT-1 or ACT-2 | ACT-1 or ACT-2 | ACT-1 or ACT-2 |
| | Medium | 62.5 μM | 125 μM | 250 μM | 500 μM | 1 mM |
| | control | | | | | |
| D | ACT-1 or ACT-2 | ACT-1 or ACT-2 | ACT-1 or ACT-2 | ACT-1 or ACT-2 | ACT-1 or ACT-2 | ACT-1 or ACT-2 |
| | Medium | 62.5 μM | 125 μM | 250 μM | 500 μM | 1 mM |
| | control | | | | | |

5. Trypsinized SVR cells (P1) and prepared 400 cells/mL solution in culture medium. Pipetted 0.5 mL cell solution to each well (200 cells/well). Mixed well by gentle pipetting.
6. Cultured cells at 37° C. incubator for 6 days.
7. Aspirated medium from the wells. Rinsed cells once with 1 mL of PBS (LONZA, Cat #: 17-516Q).
8. Fixed cells with 0.5 mL of freshly prepared 4% paraformaldehyde (PFA) (1:8 dilution of 32% PFA solution in PBS; 32% PFA solution: Electron Microscopy Sciences Cat #:15714) in PBS at RT for 20 min. Removed PFA and washed cells once with 1 mL PBS.
9. Stained cells with 0.3 mL of Mayer's hematoxylin solution (Sigma Cat#: MHS1-100ML) at RT for 15 min. Removed hematoxylin solution and rinsed cells twice with 1 mL of warm tap water.
10. Counted cell colonies under microscope with 40× magnification (divide the wells with makers into 4 sections. Count the numbers of colonies from each section and add the numbers to yield the final colony numbers). See FIG. 1.

### Results:

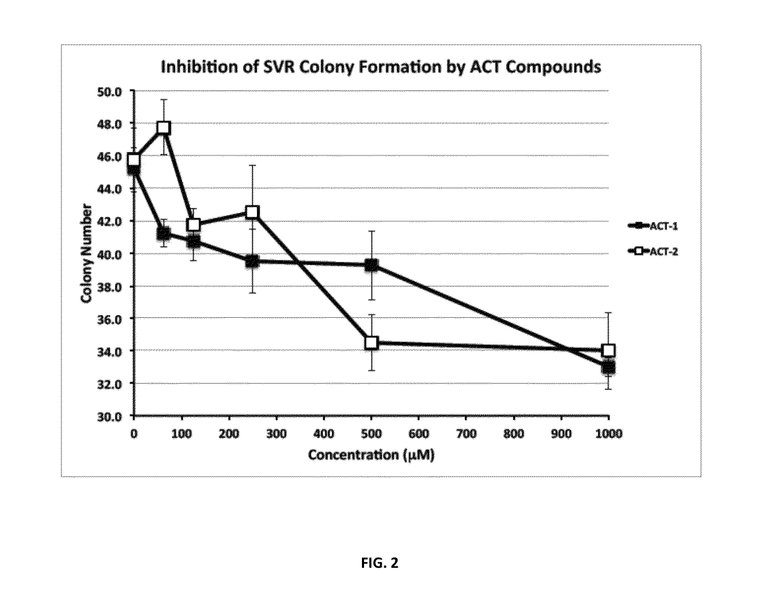

As shown in FIG. 2 as well as Tables 2 and 3, ACT-1 at 1 mM showed significant inhibition of colony formation (compared to the control, significance level P=0.001, two-tailed t-test), while ACT-2 at 0.5 mM showed significant inhibition of colony formation (compared to the control, significance level P=0.015, two-tailed t-test) (P=0.066 for 1 mM ACT-2).

TABLE 2
Colony numbers of SVR cells six days after treatment with ACT-1

| Conc. (μM) | 0 | 62.5 | 125 | 250 | 500 | 1000 |
|---|---|---|---|---|---|---|
| Well | Colony # | Colony # | Colony # | Colony # | Colony # | Colony # |
| 1 | 45 | 43 | 44 | 43 | 42 | 34 |
| 2 | 42 | 42 | 41 | 40 | 41 | 32 |
| 3 | 48 | 41 | 39 | 41 | 33 | 34 |
| 4 | 46 | 39 | 39 | 34 | 41 | 32 |
| Average | 45.3 | 41.3 | 40.8 | 39.5 | 39.3 | 33.0 |
| STDEV | 2.5 | 1.7 | 2.4 | 3.9 | 4.2 | 1.2 |
| SEM | 1.3 | 0.9 | 1.2 | 1.9 | 2.1 | 0.6 |

TABLE 3
Colony numbers of SVR cells six days after treatment with ACT-2

| Conc. (μM) | 0 | 62.5 | 125 | 250 | 500 | 1000 |
|---|---|---|---|---|---|---|
| Well | Colony # | Colony # | Colony # | Colony # | Colony # | Colony # |
| 1 | 47 | 49 | 44 | 50 | 39 | 33 |
| 2 | 47 | 51 | 42 | 44 | 31 | 36 |
| 3 | 40 | 43 | 42 | 39 | 33 | 39 |
| 4 | 49 | 48 | 39 | 37 | 35 | 28 |
| Average | 45.8 | 47.8 | 41.8 | 42.5 | 34.5 | 34.0 |
| STDEV | 3.9 | 3.4 | 2.1 | 5.8 | 3.4 | 4.7 |
| SEM | 2.0 | 1.7 | 1.0 | 2.9 | 1.7 | 2.3 |

B. The Effect of Higher Concentrations of ACT-1 and ACT-2 on the Clonogenic Survival of SVR Cells

### Experimental Procedures:

1. Testing concentrations of ACT-1 and ACT-2: 4 mM, 2 mM, 1 mM, and 0.5 mM.
2. The experimental procedures were the same as in Experiment 1-1, except that the total volume in each well was 0.6 mL (0.3 mL of 2× concentrated test articles and 0.3 mL of 667 cells/mL cell solution).

TABLE 4
Experimental layout (200 SVR cells per well)

| | 1 | 2 | 3 | 4 | 5 | 6 |
|---|---|---|---|---|---|---|
| A | ACT-1 or ACT-2 | ACT-1 or ACT-2 | ACT-1 or ACT-2 | ACT-1 or ACT-2 | ACT-1 or ACT-2 | |
| | Medium | 0.5 mM | 1 mM | 2 mM | 4 mM | |
| | control | | | | | |
| B | ACT-1 or ACT-2 | ACT-1 or ACT-2 | ACT-1 or ACT-2 | ACT-1 or ACT-2 | ACT-1 or ACT-2 | |
| | Medium | 0.5 mM | 1 mM | 2 mM | 4 mM | |
| | control | | | | | |
| C | ACT-1 or ACT-2 | ACT-1 or ACT-2 | ACT-1 or ACT-2 | ACT-1 or ACT-2 | ACT-1 or ACT-2 | |
| | Medium | 0.5 mM | 1 mM | 2 mM | 4 mM | |
| | control | | | | | |
| D | ACT-1 or ACT-2 | ACT-1 or ACT-2 | ACT-1 or ACT-2 | ACT-1 or ACT-2 | ACT-1 or ACT-2 | |
| | Medium | 0.5 mM | 1 mM | 2 mM | 4 mM | |
| | control | | | | | |

3. Cultured cells at 37° C. incubator for 6 days.
4. Visualized the cell colonies by staining with hematoxylin as described above.
5. Counted the colonies under microscope.

### Results:

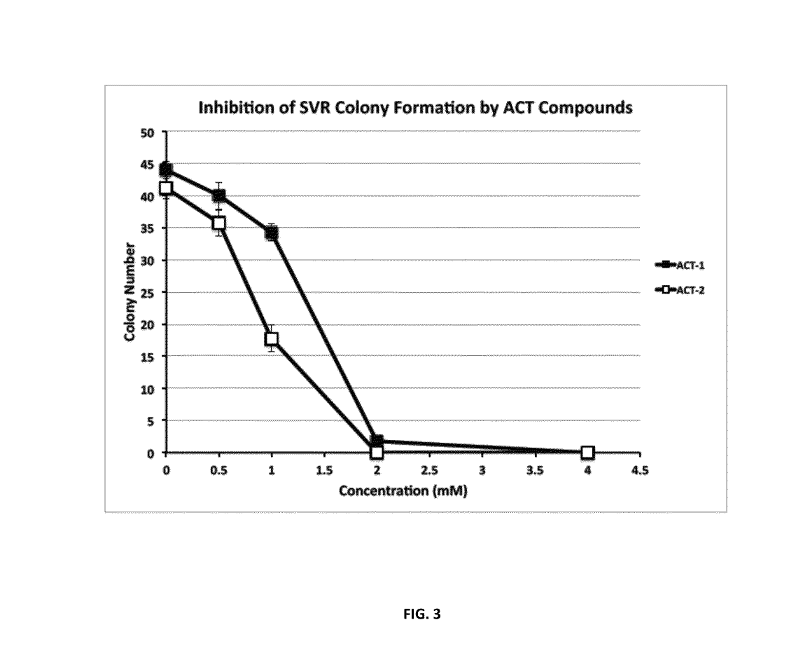

As shown in FIG. 3 and Tables 5 and 6, both ACT-1 and ACT-2 inhibited SVR cell colony formation at concentrations 1 mM and higher (significance level at P<0.05 compared to control; two-tailed t-test).

TABLE 5
Colony numbers of SVR cells six days after treatment with ACT-1

| | Conc (mM) | | | | |
|---|---|---|---|---|---|
| | 0 | 0.5 | 1 | 2 | 4 |
| Well | Colony # | Colony # | Colony # | Colony # | Colony # |
| 1 | 46 | 45 | 32 | 2 | 0 |
| 2 | 45 | 40 | 33 | 2 | 0 |
| 3 | 45 | 35 | 34 | 2 | 0 |
| 4 | 40 | 40 | 38 | 1 | 0 |
| Average | 44.0 | 40.0 | 34.3 | 1.8 | 0.0 |
| STDEV | 2.7 | 4.1 | 2.6 | 0.5 | 0.0 |
| SEM | 1.4 | 2.0 | 1.3 | 0.3 | 0.0 |

TABLE 6
Colony numbers of SVR cells six days after treatment with ACT-2

| | Conc (mM) | | | | |
|---|---|---|---|---|---|
| | 0 | 0.5 | 1 | 2 | 4 |
| Well | Colony # | Colony # | Colony # | Colony # | Colony # |
| 1 | 44 | 34 | 23 | 0 | 0 |
| 2 | 42 | 40 | 19 | 0 | 0 |
| 3 | 43 | 31 | 16 | 0 | 0 |
| 4 | 36 | 38 | 13 | 0 | 0 |
| Average | 41.3 | 35.8 | 17.8 | 0.0 | 0.0 |
| STDEV | 3.6 | 4.0 | 4.3 | 0.0 | 0.0 |
| SEM | 1.8 | 2.0 | 2.1 | 0.0 | 0.0 |

C. The Effects of ACT-1 and ACT-2 on the Apoptosis Induction of SVR Cells

### Experimental Procedures:

1. Testing concentrations of ACT-1 and ACT-2: 2 mM.
2. Prepared 4 mM ACT-1=1.82 mg/mL and 4 mM ACT-2=1.81 mg/mL in complete culture medium. Sterilized the drug solutions by filtering through 0.2 μm syringe filters.
3. Added 100 μl of test articles or culture medium to 4 wells for each ACT-1 and ACT-2 in a 96-well plate. Added 100 μl of culture medium to 5 wells (for non-treated and DNase treatment controls).
4. Trypsinized SVR cells (P3) and prepared 5×103 cells/mL solution in culture medium. Added 100 μl of cell solution to each well (500 cells/well). Mixed well by gentle pipetting.
5. Incubated at 37° C. for 24 hours.
6. Removed medium and added 100 μl of 4% PFA in PBS to the wells. Incubated at room temperature for 15 min.
7. Removed PFA solution and 100 μl of permeabilization reagent (0.25% Triton X-100 in PBS) (Triton X-100: Sigma Cat #: T8787-100ML). Incubated at room temperature for 20 min. Washed twice with deionized water.
8. TUNEL reaction was then carried out exactly as the protocol provided by the kit (Invitrogen Click-iT TUNEL Alexa Fluor Imaging Assay Kit, Cat #: C10245). Treated one well (culture medium control) with DNase as positive control for the TUNEL reaction. The reaction volume for each well was 50 μl.
9. After TdT and Click-iT reactions, DNA was stained with Hoechst 33342 (provided by the kit) 1:5,000 in PBS at RT for 15 min. Washed wells three times with PBS.
10. Observed the cells under fluorescent microscope.

### Results:

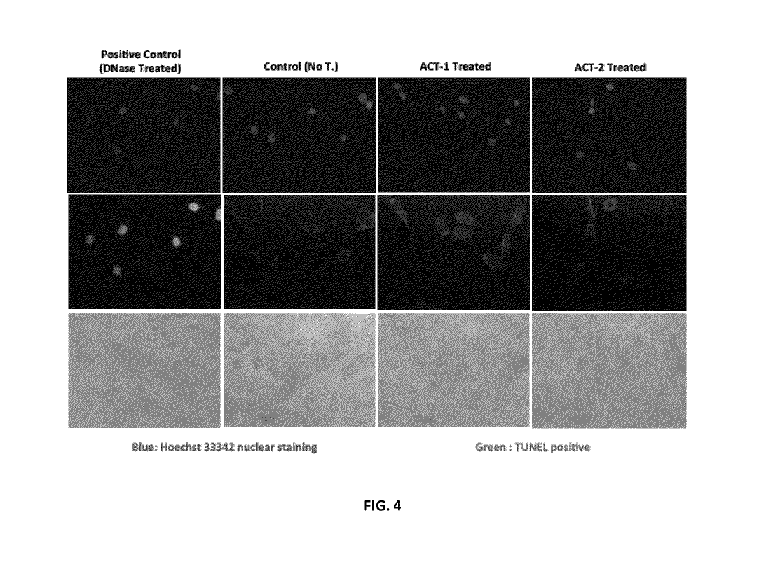

As shown in FIG. 4, nuclear staining was not observed in either control or ACT-1/ACT-2 treated cells. Cytoplasmic and some perinuclear staining was observed instead.

D. The Cytotoxic Effect of ACT-1 and ACT-2 on Primary Bovine Aortic Endothelial Cells (BAEC) and SVR Cells by MTS Assay

### Experimental Procedures:

1. Testing concentrations of ACT-1 and ACT-2: 8 mM, 4 mM, 2 mM, 1 mM, 0.5 mM, 0.25 mM and 0.125 mM.
2. Trypsinized exponentially growing BAECs (P1) and SVR cells (P4), and prepared 5×104 cells/mL cell solution for BAECs and 104 cells/mL for SVR cells. Add 100 μl of cell solution to 96-well plates to achieve 5,000 cells/well for BAECs and 1,000 cells/well for SVR cells. Incubated cells at 37° C. overnight.
3. Prepared 8 mM=3.64 mg/mL and 8 mM ACT-2=3.62 mg/mL in BAEC complete culture medium. Sterilized the drug solutions by filtering through 0.2 μm syringe filters.
4. Prepared 2× serial dilutions from 8 mM stock solution by mixing 0.6 mL drug solution with 0.6 mL culture medium.
5. Aspirated medium from wells and added 80 μl of test article solutions to the wells according to the layout below.

TABLE 7
Experimental layout (BAEC, 5,000 cells per well; SVR cells, 1,000 cells per well)

| | 1 | 2 | 3 | 4 | 5 | 6 | 7 | 8 | 9 | 10 | 11 | 12 |
|---|---|---|---|---|---|---|---|---|---|---|---|---|
| A | | Medium | Medium | Medium | Blank | Blank | Blank | | | | | |
| | | control | control | control | (no cell) | (no cell) | (no cell) | | | | | |
| B | | ACT-1 | ACT-1 | ACT-1 | ACT-2 | ACT-2 | ACT-2 | | | | | |
| | | 0.125 mM | 0.125 mM | 0.125 mM | 0.125 mM | 0.125 mM | 0.125 mM | | | | | |
| C | | ACT-1 | ACT-1 | ACT-1 | ACT-2 | ACT-2 | ACT-2 | | | | | |
| | | 0.25 mM | 0.25 mM | 0.25 mM | 0.25 mM | 0.25 mM | 0.25 mM | | | | | |
| D | | ACT-1 | ACT-1 | ACT-1 | ACT-2 | ACT-2 | ACT-2 | | | | | |
| | | 0.5 mM | 0.5 mM | 0.5 mM | 0.5 mM | 0.5 mM | 0.5 mM | | | | | |
| E | | ACT-1 | ACT-1 | ACT-1 | ACT-2 | ACT-2 | ACT-2 | | | | | |
| | | 1 mM | 1 mM | 1 mM | 1 mM | 1 mM | 1 mM | | | | | |
| F | | ACT-1 | ACT-1 | ACT-1 | ACT-2 | ACT-2 | ACT-2 | | | | | |
| | | 2 mM | 2 mM | 2 mM | 2 mM | 2 mM | 2 mM | | | | | |
| G | | ACT-1 | ACT-1 | ACT-1 | ACT-2 | ACT-2 | ACT-2 | | | | | |
| | | 4 mM | 4 mM | 4 mM | 4 mM | 4 mM | 4 mM | | | | | |
| H | | ACT-1 | ACT-1 | ACT-1 | ACT-2 | ACT-2 | ACT-2 | | | | | |
| | | 8 mM | 8 mM | 8 mM | 8 mM | 8 mM | 8 mM | | | | | |

6. Incubated cells at 37° C. for 48 hours.
7. Aspirated the medium from wells and added fresh 70 μl culture medium to the wells.
8. Prepared MTS reagent (Promega CellTiter 96 Aqueous MTS assay reagents, Cat #G5421) by mixing 2.4 mL MTS, 120 μl PMS and 3.779 mL culture medium. Added 50 μl MTS assay reagent to the wells using a multiple channel pipette.
9. Incubate at 37° C. for 2 hours. Read absorbance at 490 nm using a plate reader.

### Results:

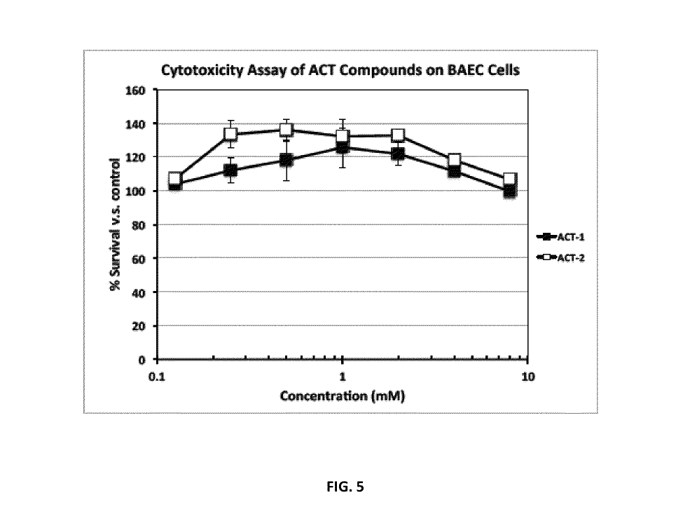

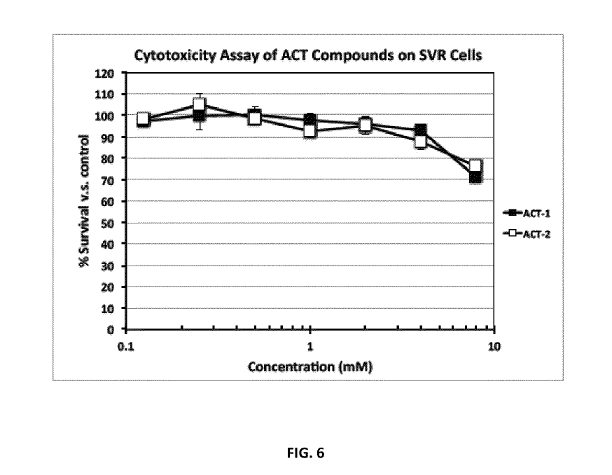

As shown in FIGS. 5 and 6 and Tables 8-11, ACT-1 and ACT-2 did not show significant cytotoxic effect on primary BAECs. ACT-1 and ACT-2 only showed significant cytotoxic effect on SVR cells at 8 mM (significance level P=0.016 for 8 mM ACT-1 and P=0.018 for 8 mM ACT-2 compared to the control by two-tailed t-test).

TABLE 8
MTS assay readings (OD 490 nm) of BAEC cells treated with ACT-1 and
ACT-2 for 48 hours.

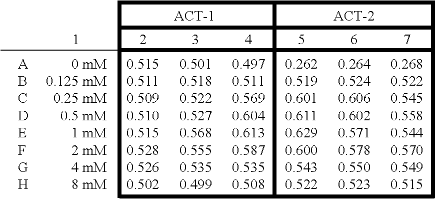

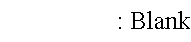

TABLE 9
MTS assay readings (OD 490 nm) of SVR cells treated with ACT-1 and
ACT-2 for 48 hours.

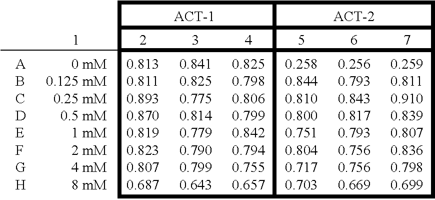

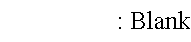

TABLE 10
Viability of BAEC cells treated with ACT-1 and ACT-2 compared
to that of the control (non-treated cells).

| Conc. | ACT-1 | | | ACT-2 | | |
|---|---|---|---|---|---|---|
| (mM) | Ave (%) | STDEV | SEM | Ave (%) | STDEV | SEM |
| 0.125 | 103.8 | 1.7 | 1.0 | 107.2 | 1.1 | 0.6 |
| 0.25 | 112.1 | 13.2 | 7.6 | 133.2 | 14.1 | 8.2 |
| 0.5 | 117.8 | 20.9 | 12.1 | 135.9 | 11.8 | 6.8 |
| 1 | 125.5 | 20.5 | 11.8 | 132.1 | 18.1 | 10.5 |
| 2 | 121.8 | 12.3 | 7.1 | 132.7 | 6.5 | 3.7 |
| 4 | 111.5 | 2.2 | 1.3 | 117.9 | 1.6 | 0.9 |
| 8 | 99.4 | 1.9 | 1.1 | 106.5 | 1.8 | 1.1 |

TABLE 11
Viability of SVR cells treated with ACT-1 and ACT-2 compared
to that of the control (non-treated cells).

| Conc. | ACT-1 | | | ACT-2 | | |
|---|---|---|---|---|---|---|
| (mM) | Ave (%) | STDEV | SEM | Ave (%) | STDEV | SEM |
| 0.125 | 97.4 | 2.4 | 1.4 | 98.2 | 4.5 | 2.6 |
| 0.25 | 99.7 | 10.8 | 6.2 | 104.9 | 9.0 | 5.2 |
| 0.5 | 100.2 | 6.6 | 3.8 | 98.7 | 3.4 | 2.0 |
| 1 | 97.7 | 5.6 | 3.2 | 92.5 | 5.1 | 3.0 |
| 2 | 95.8 | 3.2 | 1.8 | 95.1 | 7.1 | 4.1 |
| 4 | 93.1 | 4.9 | 2.8 | 87.8 | 7.1 | 4.1 |
| 8 | 71.2 | 4.0 | 2.3 | 76.1 | 3.3 | 1.9 |

### REFERENCES

1. Diehl C, Engström O, Delaine T, Håkansson M, Genheden S, Modig K, Leffler H, Ryde U, Nilsson U J, Akke M., J. Am. Chem. Soc., 2010, 132(41), 14577-89.
2. Nilsson U., Leffler H., Cumpstey I., U.S. Pat. No. 7,638,623 B2, 2009.
3. Johnson K D, Glinskii O V, Mossine V V, Turk J R, Mawhinney T P, Anthony D C, Henry C J, Huxley V H, Glinsky G V, Pienta K J, Raz A, Neoplasia, 2007, 9(8), 662-70.
4. Glinsky V V, Kiriakova G, Glinskii O V, Mossine V V, Mawhinney T P, Turk J R, Glinskii A B, Huxley V H, Price J E, Glinsky G V, Neoplasia, 2009, 11(9), 901-9.
5. Glinskii G V, U.S. Pat. No. 5,864,024, 1999.
6. Glinskii G V, U.S. Pat. No. 5,629,412, 1997.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound of Formula I 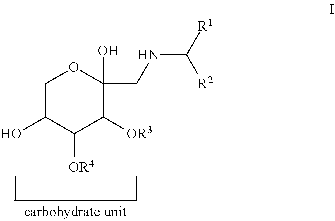
carbohydrate unit
or a pharmaceutically acceptable salt thereof,
wherein:
R¹ is selected from the group consisting of: H, CO₂H, C(O)NH₂, C(O)NHOH, C(O)NHOR⁵, CO₂R⁶, C(O)NHR⁷, C(O)NR⁸R⁹, heterocyclyl, and heteroaryl, wherein R⁵, R⁶, R⁷, R⁸, and R⁹ are independently selected from the group consisting of: C₁-C₆ alkyl, carbocyclyl, heterocyclyl, aryl, and heteroaryl, or R⁸ and R⁹ can combine with the N atom to which they are attached to form a 5 or 6-membered ring, or NHR⁷ is a normatural α-amino acid or a normatural peptide;
R² is selected from the group consisting of: C₁-C₆ alkyl, C₁-C₆ haloalkyl, C₁-C₆ hydroxyalkyl, C₁-C₆ alkoxy, C₂-C₆ alkenyl, C₂-C₆ alkynyl, C₃-C₈ carbocyclyl, heterocyclyl, aryl, and heteroaryl;
wherein if R¹ is CO₂H, then the —NHCH(R²)CO₂H moiety on the compound of Formula I forms a normatural α-amino acid;
wherein if R¹ is H, then R² is selected from the group consisting of C₃-C₈ carbocyclyl, benzyl, heterocyclyl, aryl, and heteroaryl;
wherein the above alkyl, alkenyl, alkynyl, carbocyclyl, heterocyclyl, aryl, and heteroaryl moieties are each optionally and independently substituted by 1-3 substituents selected from the group consisting of: amino, cyano, halo, hydroxyl, nitro, C₁-C₆ alkylamine, C₁-C₆ dialkylamine, C₁-C₆ alkyl, C₁-C₆ alkoxy, C₂-C₆ alkenyl, and C₁-C₆ hydroxyalkyl;
R³ and R⁴ are each independently selected from H and a monosaccharide, provided only one of R³ and R⁴ can be a monosaccharide.

2. The compound of claim 1, wherein the carbohydrate unit is a natural or modified sugar.

3. The compound of claim 2, wherein the sugar is a monosaccharide.

4. The compound of claim 3, wherein the monosaccharide is selected from the group consisting of: arabinose, xylose, ribose, ribulose, fructose, deoxyfructose, galactose, glucose, mannose, tagatose, and rhamnose.

5. The compound of claim 2, wherein the sugar is a disaccharide.

6. The compound of claim 5, wherein the disaccharide is selected from the group consisting of: lactulose, lactose, maltulose, and maltose.

7. The compound of claim 2, wherein each of the hydroxyl groups can be independently protected by a protecting group.

8. The compound of claim 1, wherein the compound is optically pure.

9. The compound of claim 1, wherein R¹ is selected from the group consisting of: H, CO₂H, C(O)NH₂, C(O)NHOH, C(O)NHOR⁵, CO₂R⁶, C(O)NHR⁷, C(O)NR⁸R⁹, heterocyclyl, and heteroaryl; wherein R⁵, R⁶, R⁷, R⁸, and R⁹ are independently C₁-C₆ alkyl, carbocyclyl, heterocyclyl, aryl, and heteroaryl.

10. The compound of claim 9, wherein R¹ is C(O)NHR⁷, wherein NHR⁷ is an normatural α-amino acid or normatural peptide.

11. The compound of claim 9, wherein R¹ is selected from the group consisting of: CO₂H, CO₂Me, CO₂Et, C(O)NH₂, C(O)NHOH, C(O)NHMe, and C(O)NH(Me)₂.

12. The compound of claim 1, wherein R² is selected from the group consisting of C₁-C₆ alkyl, C₂-C₆ alkenyl, C₂-C₆ alkynyl, C₁-C₆ haloalkyl, C₃-C₈ carbocyclyl, heterocyclyl, aryl, and heteroaryl.

13. The compound of claim 12, wherein R² is selected from the group consisting of C₁-C₆ alkyl, C₃-C₈ carbocyclyl, heterocyclyl, aryl, and heteroaryl.

14. The compound of claim 12, wherein R¹ is H, and R² is selected from the group consisting of: C₃-C₈ carbocyclyl, substituted or unsubstituted benzyl, heterocyclyl, aryl, and heteroaryl.

15. The compound of claim 12, wherein R¹ is CO₂H, and the —NHCH(R²)CO₂H moiety on the compound of Formula I is a normatural α-amino acid.

16. The compound of claim 15, wherein R² is selected from the group consisting of:

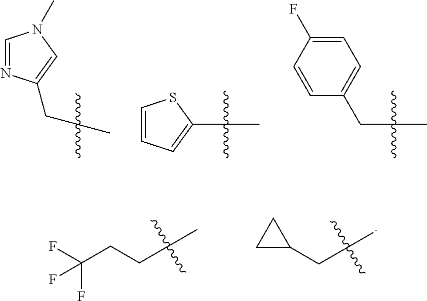

17. A compound selected from the group consisting of:
3-(3-Methyl-3H-imidazol-4-yl)-2-{[2,3,5-tri hydroxy-4-(3,4,5-trihydroxy-6-hydroxymethyl-tetrahydro-pyran-2-yloxy)-tetrahydro-pyran-2-ylmethyl]-amino}-propionic acid; 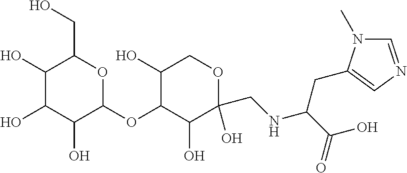 [SMILES: Cn1cncc1CC(NCC1(O)OCC(O)C(OC2OC(CO)C(O)C(O)C2O)C1O)C(=O)O]
Thiophen-2-yl-{[2,3,5-trihydroxy-4-(3,4,5-trihydroxy-6-hydroxymethyl-tetrahydro-pyran-2-yloxy)-tetrahydro-pyran-2-ylmethyl]-amino}-acetic acid; 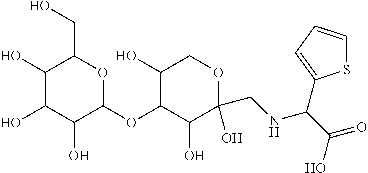 [SMILES: O=C(O)C(NCC1(O)OCC(O)C(OC2OC(CO)C(O)C(O)C2O)C1O)c1cccs1]
3-(4-Fluoro-phenyl)-2-{[2,3,5-trihydroxy-4-(3,4,5-trihydroxy-6-hydroxymethyl-tetrahydro-pyran-2-yloxy)-tetrahydro-pyran-2-ylmethyl]-amino}-propionic acid; 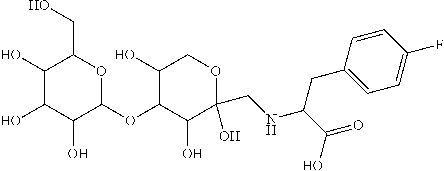 [SMILES: O=C(O)C(Cc1ccc(F)cc1)NCC1(O)OCC(O)C(OC2OC(CO)C(O)C(O)C2O)C1O]
5,5,5-Trifluoro-2-{[2,3,5-trihydroxy-4-(3,4,5-trihydroxy-6-hydroxymethyl-tetrahydro-pyran-2-yloxy)-tetrahydro-pyran-2-ylmethyl]-amino}-pentanoic acid; 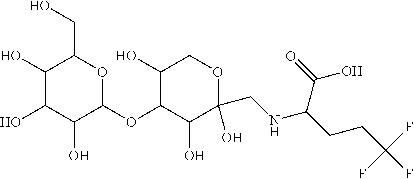 [SMILES: O=C(O)C(CCC(F)(F)F)NCC1(O)OCC(O)C(OC2OC(CO)C(O)C(O)C2O)C1O]
3-Cyclopropyl-2-{[2,3,5-trihydroxy-4-(3,4,5-trihydroxy-6-hydroxymethyl-tetrahydro-pyran-2-yloxy)-tetrahydro-pyran-2-ylmethyl]-amino}-propionic acid; 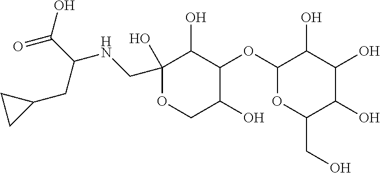 [SMILES: O=C(O)C(CC1CC1)NCC1(O)OCC(O)C(OC2OC(CO)C(O)C(O)C2O)C1O]
3-Cyclopropyl-2-{[2,3,5-trihydroxy-4-(3,4,5-trihydroxy-6-hydroxymethyl-tetrahydro-pyran-2-yloxy)-tetrahydro-pyran-2-ylmethyl]-amino}-propionic acid methyl ester; 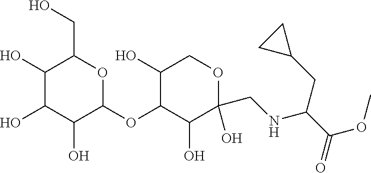 [SMILES: COC(=O)C(CC1CC1)NCC1(O)OCC(O)C(OC2OC(CO)C(O)C(O)C2O)C1O]
3-Cyclopropyl-2-{[2,3,5-trihydroxy-4-(3,4,5-trihydroxy-6-hydroxymethyl-tetrahydro-pyran-2-yloxy)-tetrahydro-pyran-2-ylmethyl]-amino}-propionamide; 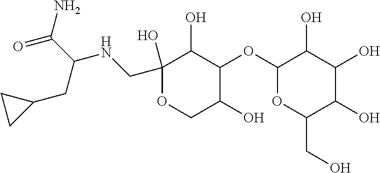 [SMILES: NC(=O)C(CC1CC1)NCC1(O)OCC(O)C(OC2OC(CO)C(O)C(O)C2O)C1O]
(4-Fluoro-phenyl)-{[2,3,5-trihydroxy-4-(3,4,5-trihydroxy-6-hydroxymethyl-tetrahydro-pyran-2-yloxy)-tetrahydro-pyran-2-ylmethyl]-amino}-methane; 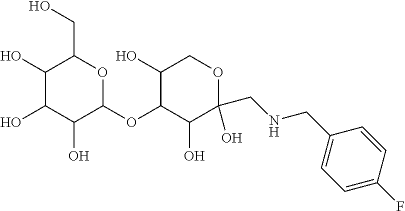 [SMILES: OCC1OC(OC2C(O)COC(O)(CNCc3ccc(F)cc3)C2O)C(O)C(O)C1O]
Cyclopropyl-{[2,3,5-tri hydroxy-4-(3,4,5-trihydroxy-6-hydroxymethyl-tetrahydro-pyran-2-yloxy)-tetrahydro-pyran-2-ylmethyl]-amino}-ethane 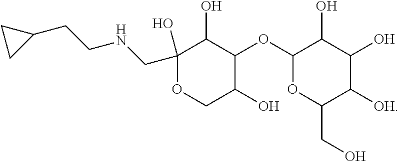 [SMILES: OCC1OC(OC2C(O)COC(O)(CNCCC3CC3)C2O)C(O)C(O)C1O]
or a pharmaceutically acceptable salt thereof.

18. A pharmaceutical composition comprising a compound of claim 1, or a pharmaceutically acceptable salt thereof, and a pharmaceutically acceptable carrier.

19. A method of affecting cell adhesion and inducing apoptosis in a patient, the method comprising administering to the patient a therapeutically effective amount of a compound of claim 1, or a pharmaceutically acceptable salt thereof.

20. A method of inhibiting galectin-3 in a patient, the method comprising administering to the patient a therapeutically effective amount of a compound of claim 1, or a pharmaceutically acceptable salt thereof.

21. A method of treating metastatic diseases and cancer in a patient in need thereof, the method comprising administering to the patient a therapeutically effective amount of a compound of claim 1, or a pharmaceutically acceptable salt thereof.

22. The method of claim 21, wherein the patient is a human.

23. The method of claim 21 further comprising administering an additional therapeutic agent to the patient.

24. The method of claim 23, wherein the additional therapeutic agent is selected from the group consisting of: antibiotics, antiemetic agents, antidepressants, antifungal agents, anti-inflammatory agents, antiviral agents, and anticancer agents.

25. The method of claim 24 wherein the additional therapeutic agent is an anti-cancer agent.
